(19) United States
(12) Patent Application Publication
(10) Pub. No.: US 2022/0151631 A1
(43) Pub. Date: May 19, 2022

(54) Apparatus and Methods for Intravascular Treatment of Aneurysms

(71) Applicant: MG Stroke Analytics Inc., Calgary (CA)

(72) Inventor: Mayank Goyal, Calgary (CA)

(21) Appl. No.: 17/645,811

(22) Filed: Dec. 23, 2021

Related U.S. Application Data
(62) Division of application No. 16/239,296, filed on Jan. 3, 2019, now abandoned.
(60) Provisional application No. 62/616,980, filed on Jan. 12, 2018.

(51) Int. Cl. A61B 17/12

(52) U.S. Cl. CPC A61B 17/12113; A61B 2017/12054; A61B 17/12031; A61B 17/12168

(57) ABSTRACT

The invention relates to the treatment of aneurysms, and more particularly to intravascular devices and methods for the occlusion of an aneurysm. The device includes a first portion having an expandable and compressible mesh having dimensions for insertion into and expansion against the wall of an aneurysm and a second disk portion having a flexible, collapsible mesh operatively connected to an outer surface of the first portion and having dimensions for covering an outside of the neck opening. The combination of the first portion and second disk portion have a combined resilient flexibility to effectively bias the second disk portion against the neck opening in a substantially flat manner when the first portion is engaged within the aneurysm.

### FIELD OF THE INVENTION

The invention relates to the treatment of aneurysms, and more particularly to intravascular devices and methods thereof for treating intracranial aneurysms.

### BACKGROUND

An aneurysm is a blood-filled balloon-like bulge in the wall of a blood vessel, typically caused by flowing blood forcing a weakened section of the blood vessel wall outwards. Aneurysms can occur in any blood vessel but can be particularly problematic when they occur in a cerebral artery. Known as an intracranial or cerebral or brain aneurysm, if a brain aneurysm ruptures, it can lead to a hemorrhagic stroke and potentially cause death or severe disability. The risk of rupture increases with the size of the aneurysm. Most people with un-ruptured brain aneurysms do not have any symptoms and the aneurysm goes undetected. If the aneurysm is by chance detected, which often occurs incidentally, it may be desirable to treat the aneurysm to prevent it from growing, thereby reducing the risk of rupture.

When a patient presents to the hospital with a ruptured brain aneurysm: known as sub-arachnoid hemorrhage (SAH), it is a serious medical emergency. Ruptured aneurysms have a high likelihood of re-rupture which can have devastating consequences. As such, ruptured aneurysms need to be treated as a surgical emergency.

Brain aneurysms 10 develop in various shapes and sizes as shown in FIGS. 1A-1C with each aneurysm generally characterized by a neck 12 that opens from an artery 14 into an enlarged capsular structure or body. An aneurysm generally has a neck diameter ND, internal radius R and neck angle NA. FIGS. 1A (side view) and 1AA (end view) show the most common type namely a saccular aneurysm that is a “berry-like” bulge or sac that occurs in an artery. In this example, the neck diameter is relatively small compared to the internal radius and the neck angle is less than 90 degrees. FIG. 1B shows a different aneurysm structure having a less spherical shape and that is characterized by a wider neck and a neck angle around 90 degrees. FIG. 10 shows an aneurysm structure where the neck diameter is also greater relative to the internal radius and the neck angle is greater than 90 degrees on at least one side of the aneurysm. Variations in these general types include eccentrically inclined aneurysms (not shown). As will be discussed in greater detail below, the treatment of each of these aneurysms is different.

Generally, the size of the neck typically varies from 2-7 mm and the internal diameter (2 times internal radius) may vary from 3-8 mm. Some aneurysms may also have an irregular protrusion of the wall of the aneurysm, i.e. a “daughter sac”.

The size, shape and location of a brain aneurysm influences the availability and type of treatment. Historically, some brain aneurysms were treated surgically by clipping or closing the base or neck of the aneurysm. Due to the risks and invasiveness of open brain surgery, treatment has moved towards less invasive intravascular techniques. With intravascular techniques, a microcatheter is inserted into the arterial system of a patient, usually through the groin, and threaded through the arterial system to the site of the aneurysm. With one technique, as shown in FIG. 2A, a wire 15 is pushed from a microcatheter 16 and coiled into the body of the aneurysm, in order to pack the aneurysm body with a coil of wire. This wire coil 15 is subsequently detached from the microcatheter by known techniques to enable the microcatheter and remaining wire within the microcatheter to be withdrawn. The wire coil prevents or slows the flow of blood into the aneurysm, causing a thrombus to form in the aneurysm and which then ideally prevents the aneurysm from growing and/or rupturing. During placement and subsequently, it is important that the coil stays within the aneurysm body and does not protrude into the artery. Therefore, this endovascular coiling technique, works best in aneurysms that have narrow necks as shown in FIG. 1A and more specifically with neck diameters less than approximately <4 mm in order to keep the coiled wire within the aneurysm body.

In aneurysms with slightly wider necks, that is, similar to an aneurysm as shown in FIG. 1B, balloon-assisted coiling may be used to prevent the coil from protruding into the artery. As shown in FIGS. 2B-2E, a first catheter 16 containing a wire 15 is inserted into the aneurysm body 10. A second catheter 18 having a balloon 20 is placed in the artery adjacent the neck 12 of the aneurysm. As the wire 15 is coiled into the aneurysm, the balloon 20 is temporarily inflated to keep the coiled wire 15 within the aneurysm body. After coiling is complete, or after enough wire has been coiled to keep the wire in place, the balloon is deflated and removed from the artery. One of the risks associated with this type of procedure is that the microcatheter may be too rigid because of the pressure from the balloon and hence may cause the aneurysm to rupture. Other risks are the presence of an inflated balloon in the parent vessel that can lead to thrombus formation. Rarely the vessel may rupture because of overinflation of the balloon. Most importantly, there is a chance that the coils may prolapse out of the aneurysm once the balloon has been deflated.

In another approach called stent assisted coiling, a stent is placed into the parent vessel preventing the prolapse of the coils. This approach has some of the disadvantages of balloon assisted coiling but in addition, the other problem is that stents are quite thrombogenic and hence, patients need to be placed on blood-thinners in preparation for stent placement. Of note, some patients have resistance to different blood thinners further adding to the complexity. In addition, and generally speaking, it is difficult to use stent assisted coiling in acutely ruptured aneurysms as there isn't sufficient time for the blood thinners to act and in addition blood thinners may not be safe in the presence of SAH.

In another endovascular treatment option, instead of a coiled wire, a pre-formed and compressed/collapsed wire mesh ball 22 is pushed out of the catheter and deployed into the body of the aneurysm 10 as shown in FIG. 3A. In this case, the physician chooses a mesh ball size that will best fit within the aneurysm when expanded. Generally, preformed and compressed wire mesh balls are spherical and have specific diameters that can fit within an aneurysm. When deployed and detached, like the coiled wire, the mesh ball seals and/or prevents or slows the flow of blood into the aneurysm, causing a thrombus to form in the aneurysm. This approach typically works best in aneurysms that are more spherical in shape and have a narrow neck to keep the mesh ball within the aneurysm body. However, as shown in FIG. 3B, if the neck is wide and the mesh ball is substantially spherical, regions of the aneurysm may not be completely filled which can result in unfilled pockets 10a, 10b such that if turbulent blood flow is created in those regions, it can result in growth of the aneurysm. In addition, there is also a possibility of aneurysm rupture or thrombus formation that can subsequently break away and cause stroke.

In another intravascular treatment approach for aneurysms as shown in FIG. 4A, a tubular stent 24, i.e. a metal mesh device in the shape of a tube, is placed inside the artery at the site of the aneurysm to cover the neck of the aneurysm. The stent blocks the flow of blood into the aneurysm, allowing a thrombus to form in the aneurysm. Often the aneurysm will shrink over time after the stent is in place. A stent 24 is particularly useful for large aneurysms and/or aneurysms with wide necks and/or irregular shaped bodies. A stent may be used on its own or in conjunction with another device like a coiled wire or mesh ball. The stent can help keep the coiled wire or mesh ball within the aneurysm body if the aneurysm has a wide neck. The disadvantages of a stent are that it creates a large area of metal within the artery which increases the chance of thrombi forming on the stent. Patients with stents typically need to take antiplatelet medication indefinitely to prevent blood clots from forming and growing. While stents can work well for certain types of aneurysms, particularly ones that are located in straight arterial passageways, they are not ideal for all aneurysms. That is, if there are one or more bifurcations 14a in the arterial vessel near the aneurysm, the stent would block off flow to the other vessel and would therefore not be suitable for use if the aneurysm is located near a bifurcation 14a as shown in FIG. 4B.

Another recently developed device for treating brain aneurysms is an endovascular clip system, referred to as an eCLIP™, shown in FIG. 5. The eCLIP™ is a stent-like metal device that is guided intravascularly to the site of the aneurysm. Unlike a stent, it does not cover the entire circumference of the blood vessel, but only approximately half of the circumference. The eCLIP™ has a first segment 30 with more densely packed “arms” that cover the neck of the aneurysm to block or slow the flow of blood into the aneurysm. There is a second segment 32 that has less densely packed arms that serves as an anchor to keep the eCLIP™ in place in the blood vessel. The eCLIP™ is particularly useful for an aneurysm 10 having a wide neck 12 where there are one or more bifurcations 34 on the side of the vessel opposite the aneurysm, as shown in FIG. 5. However, this device does not address the situation of one or more bifurcations on the same side as the aneurysm as shown in FIG. 4B where placement would occlude a vessel. Generally speaking, this device has been found to be extremely difficult to use and has so far not been successful.

In addition, systems have been proposed incorporating various designs of covers that when deployed cover a neck opening. These include various designs that include systems for covering at least part of a neck opening and that may be held in position by both internal and external system.

Examples of a number of different aneurysm treatment systems including wire coils, neck covers, external stent supports and others are described in U.S. Pat. Nos. 6,506,204, 6,592,605, 6,936,055, 8,062,379, 8,075,585, 8,388,650, 8,444,667, 8,529,556, 8,545,530, 8,551,132, 8,668,716, 8,715,312, 8,876,863, 8,979,893, 9,034,054, 9,089,332, 9,119,625, 9,259,337, 9,277,924, US Patent 2016/0249937, US Patent Publication 2004/0111112, US Patent Publication 20130304109, US Patent Publication 2012/0143317, US Patent Publication 2008/0221600, US Patent Publication 2007/0203452, US Patent Publication 2007/0198075, US Patent Publication 2007/0106311, US Patent Publication 2003/0195553, U.S. Pat. Nos. 8,926,681, 7,621,928, 7,232,461, 6,663,607, 6,454,780, 6,383,174, 6,361,558, 6,309,367, 6,093,199, 6,063,104, 7,744,652, 7,195,636 and 5,951,599.

While these systems are examples of a wide variety of aneurysm treatment systems, there continues to be a need for improved systems and methods for treating brain aneurysms, particularly ones that are irregularly shaped and/or have wide necks. There is also been a need for neck cover systems having increased flexibility in the types of neck openings that can be treated and particularly systems where individual neck covering leaflets or leaves can move relative to one another.

### SUMMARY

In a first aspect, the invention provides an occlusion device for inserting into an aneurysm to occlude blood flow into the aneurysm where the aneurysm has a neck opening and a plurality of walls adjacent the neck opening. The device includes a first portion having an expandable and compressible mesh having dimensions for insertion into and expansion against the aneurysm walls; a second disk portion having a flexible, collapsible mesh operatively connected to an outer surface of the first portion and having dimensions for covering an outside of the neck opening where the combination of the first portion and second disk portion have a combined resilient flexibility to effectively bias the second disk portion against the neck opening in a substantially flat manner when the first portion is engaged within the aneurysm.

In various embodiments, the device is reversibly collapsible and expandable into and from a microcatheter and/or the device is selectively detachable from a microwire within the microcatheter.

Generally, the first portion may be a sphere, ellipsoid or partial/half sphere/ellipsoid.

In one embodiment, the first portion has a central connection point and a plurality of radial segments and the radial segments can independently flex relative to each other about a central core.

In a further embodiment, the second portion is circular.

In further embodiments, the second disk portion has a central core and a plurality of radial segments where the central core has dimensions to substantially cover the neck opening and the radial segments can independently flex relative to each other about the central core and/or the second disk portion has sufficient flexibility to effectively conform the second disk portion to the inner shape of an artery in which it is deployed.

In one embodiment, the second portion is collapsible within a microcatheter in an inverted position.

In one embodiment, the second portion includes a plurality of radial segments operative connected to a connection point and where each radial segment has a flexure zone adjacent the connection point having a shape-memory to bias each radial segment in a position upward of a plane tangential to a base of the first portion. The flexure zone enables each radial segment to be loaded into a catheter with the radial segments oriented in a proximal direction and when loaded each radial segment is biased against an inner wall of the catheter and where upon deployment of the occlusion device from the catheter, the flexure zone of each radial segment biases the radial segments to the extended position.

In one embodiment, the connection point is a sleeve having a proximal end and distal end and the first portion and second portion are secured to the connection point through the distal end so as to extend distally from the connection point.

In another aspect, the invention provides a kit for enabling a medical procedure to treat an aneurysm comprising an occlusion device operatively connected to a microwire and operatively collapsed within a microcatheter.

In another aspect, the invention provides a method of deploying an occlusion device within an aneurysm having a neck opening, the occlusion device operatively connected to a microwire and operatively contained within a microcatheter adjacent a distal tip of the microcatheter, the method comprising the steps of:

a) advancing the microcatheter through a patient's vasculature to the aneurysm;
b) manipulating the distal tip into the neck opening;
c) withdrawing the microcatheter while maintaining forward pressure on the microwire to deploy the first portion into the aneurysm;
d) further withdrawing the microcatheter while maintaining forward pressure on the microwire to deploy the second portion over the neck opening of the aneurysm;
e) detaching the microwire from the occlusion device; and,
f) withdrawing the microcatheter and microwire from the patient's vasculature.

### BRIEF DESCRIPTION OF THE DRAWINGS

Various objects, features and advantages of the invention will be apparent from the following description of particular embodiments of the invention, as illustrated in the accompanying drawings. The drawings are not necessarily to scale, emphasis instead being placed upon illustrating the principles of various embodiments of the invention. Similar reference numerals indicate similar components.

FIGS. 1A, 1AA, 1B and 1C are schematic diagrams of different aneurysm structures showing typical variations in neck diameter and neck angle.

FIGS. 2A-2E are schematic diagrams of wire coiling methodologies for treating aneurysms including narrow neck and wider neck aneurysms with a balloon catheter (FIGS. 2B-2D) and without a balloon catheter (FIG. 2A) in accordance with the prior art.

FIGS. 3A and 3B are schematic diagrams showing the methodology of placing and deploying a wire mesh ball for the treatment of an aneurysm in accordance with the prior art.

FIGS. 4A and 4B are schematic diagrams showing a methodology of placing a wire mesh stent for the treatment of an aneurysm away from a bifurcation (FIG. 4A) and near a bifurcation (FIG. 4B) in accordance with the prior art.

FIG. 5 is a schematic diagram of an endovascular clip system for the treatment of a brain aneurysm and its placement near arterial bifurcations in accordance with the prior art.

FIGS. 6A-6C are a schematic cross-sectional side view, cross-sectional end view and top view respectively of an occlusion device deployed in an aneurysm in accordance with one embodiment of the invention.

FIG. 6D is a schematic bottom view of an occlusion device having a segmented second portion in accordance with one embodiment of the invention.

FIG. 6E is a schematic bottom view of an occlusion device having a segmented second portion having spaces between segments in accordance with one embodiment of the invention.

FIG. 6F is a schematic side view of an occlusion device having a segmented second portion in accordance with one embodiment of the invention fit within an aneurysm and showing how segments may flex with respect to an artery wall.

FIG. 6G is a schematic side view of an occlusion device having a segmented second portion in accordance with one embodiment of the invention shown in a relaxed position with upwardly/downwardly biased segment arms.

FIG. 6H is a schematic three-dimensional view of an occlusion device having a segmented second portion in accordance with one embodiment of the invention.

FIG. 6I is a schematic cross-sectional side view of an occlusion device having a partial-sphere or segmented first portion shown deployed in an aneurysm in accordance with one embodiment of the invention.

FIG. 6J is a schematic plan view of an occlusion device having a segmented first and segmented second portion in accordance with one embodiment of the invention.

FIG. 6K is a schematic plan view of an occlusion device having a segmented second portion in accordance with one embodiment of the invention having 8 overlapping leaflets.

FIG. 6L is a schematic plan view of an occlusion device having a segmented second portion in accordance with one embodiment of the invention having 4 overlapping leaflets.

FIG. 6M is a schematic side view of an occlusion device in accordance with the invention showing additional tubular stents deployed.

FIGS. 6N (small scale) and 6O (large scale) are schematic sectional views of an occlusion device showing a mechanism of attaching a second portion to a central portion with a flexure zone biasing the second portion to an upward position. For clarity these figures are shown as sections about a centerline.

FIGS. 6P (large scale) and 6Q (small scale) are schematic sectional views of an occlusion device showing a mechanism of attaching a second portion to a central portion where the connection point is sleeve that biases the second portion to an upward position. For clarity these figures are shown as sections about a centerline.

FIGS. 7A to 7D are cross-sectional side views of an occlusion device being deployed at the site of an aneurysm in accordance with one embodiment of the invention.

FIGS. 8A-8C are cross-sectional views of the deployment and recovery of an occlusion device from and into a microcatheter in accordance with one embodiment of the invention.

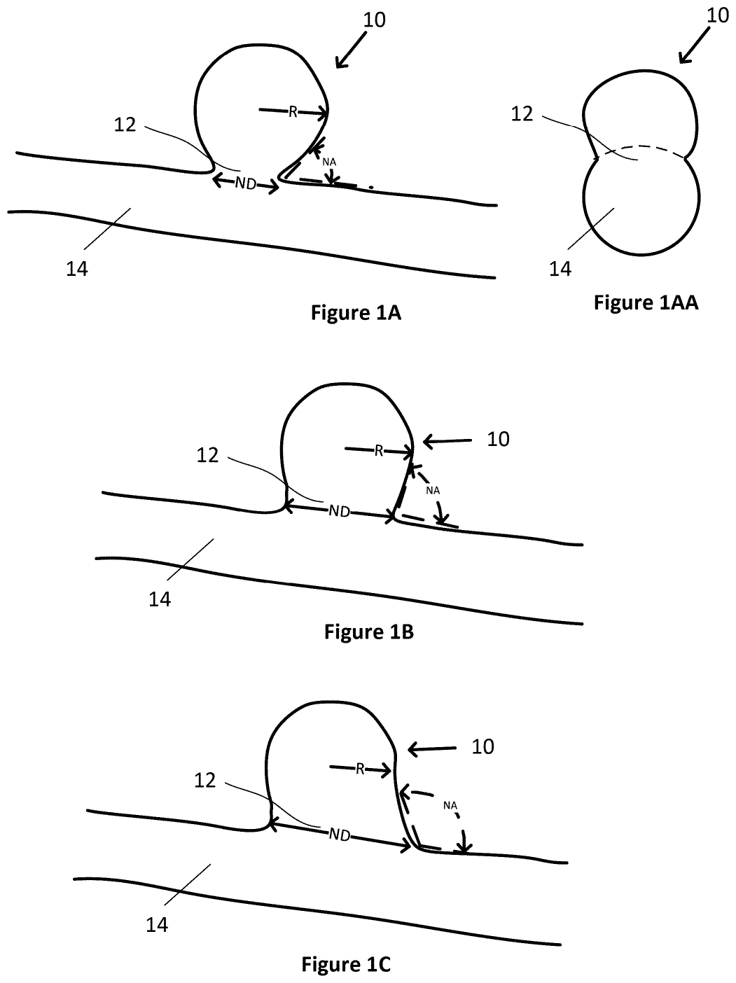

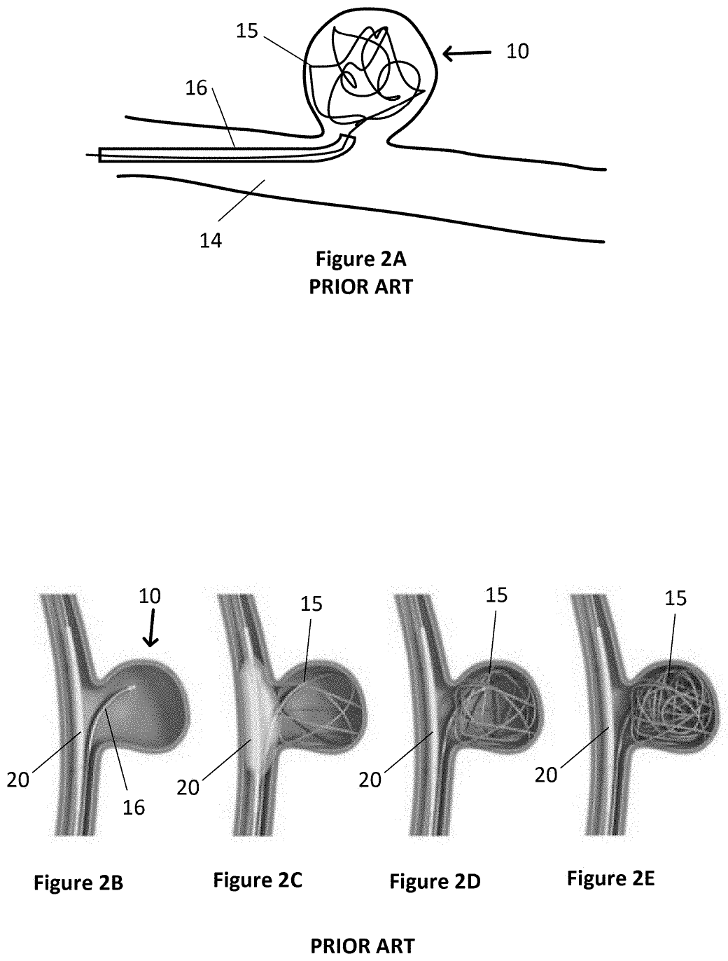

### DETAILED DESCRIPTION

With reference to the figures, devices and methods for the intravascular treatment of aneurysms are described. More specifically, occlusion devices for deployment at the site of aneurysms to limit blood from flowing into the aneurysms and methods of deployment using the intravascular system are described. The embodiments described in the figures are not necessarily drawn to scale and are intended to show general principles of design and deployment of the invention. Variations in the relative dimensions can be made in accordance with the performance and operational objectives described herein.

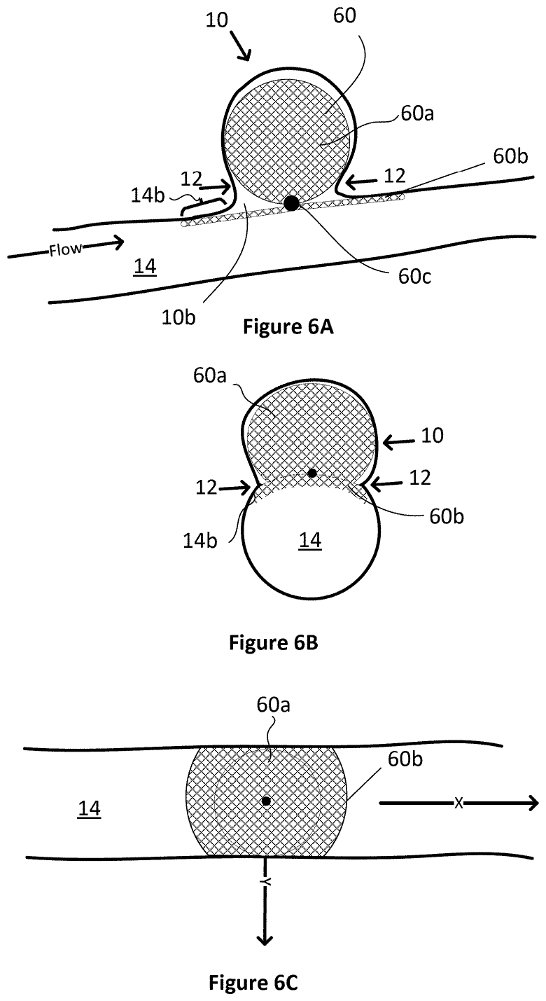

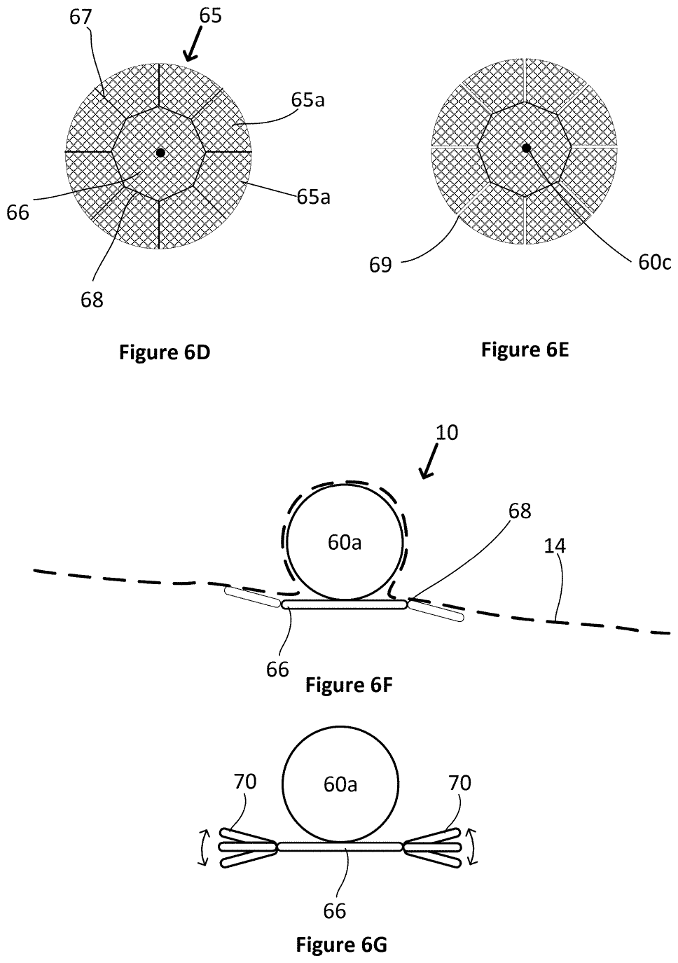

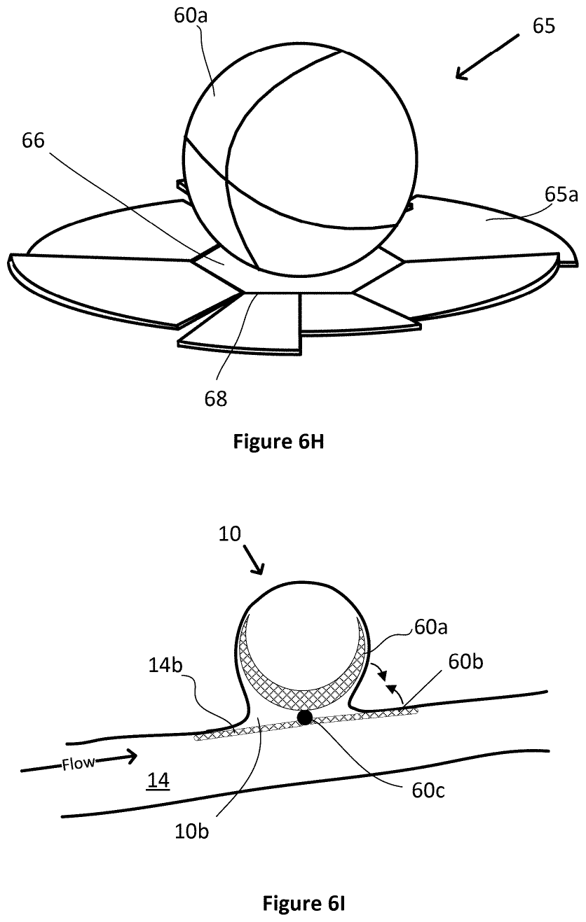

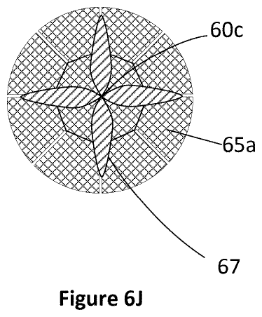

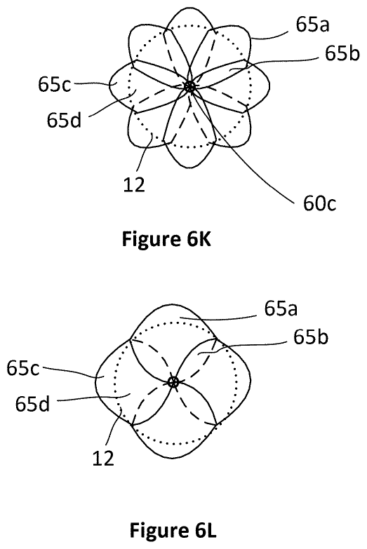

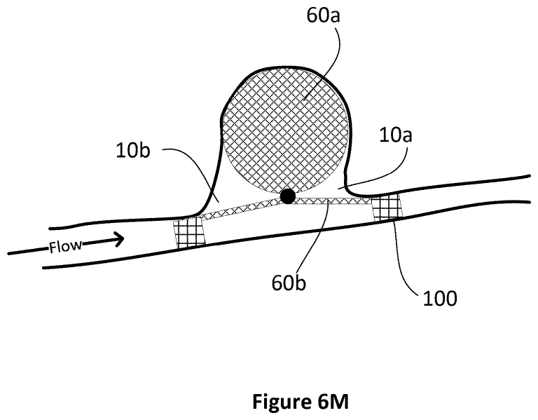

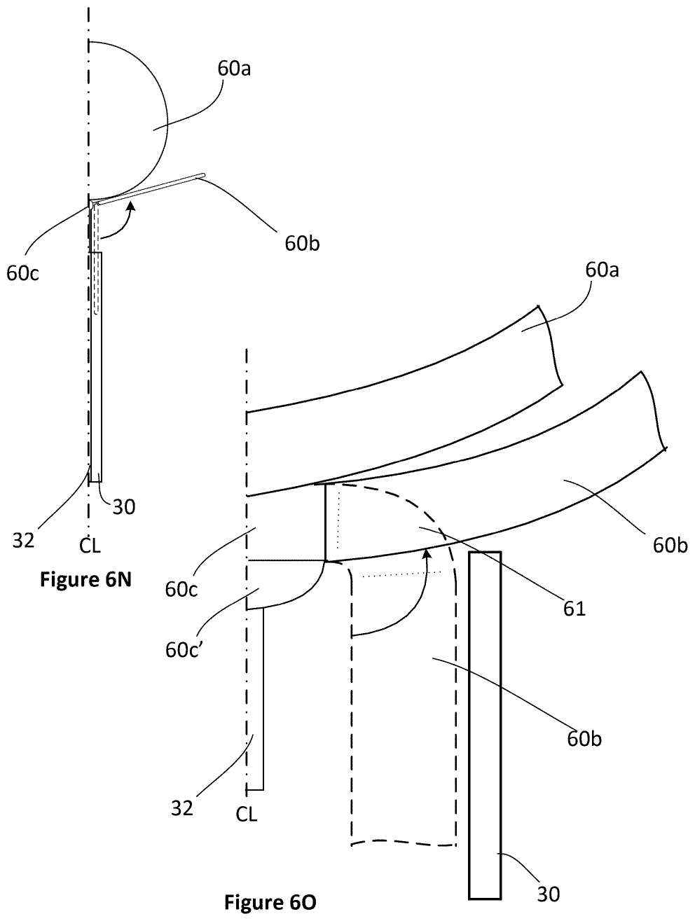

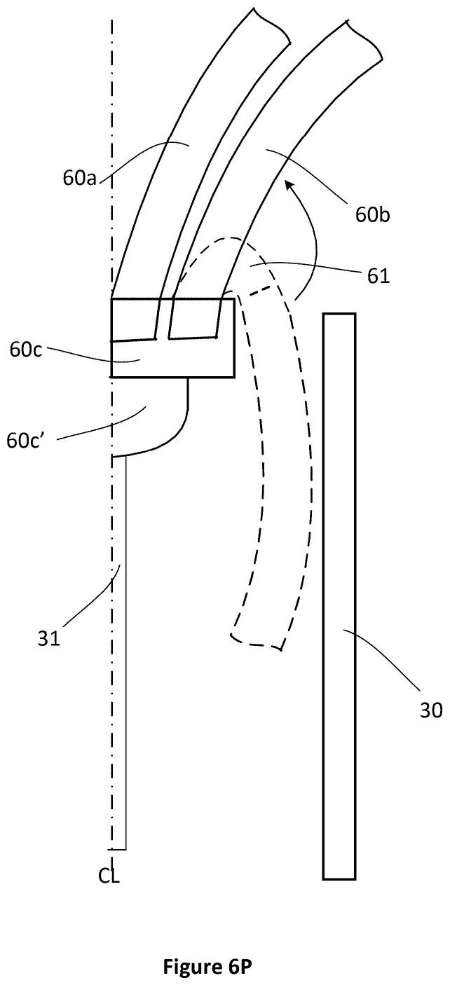

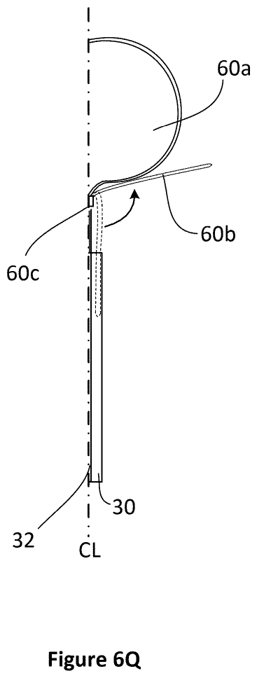

For the purposes of context, the following description is made with reference to brain aneurysms although it is understood that the devices and methodologies described are applicable to other aneurysms. FIGS. 6A-6C illustrate a cross-sectional side view, end view and bottom view, respectively, of an aneurysm 10 within an intracranial artery 14. An occlusion device 60 has been deployed at the site of the aneurysm, the device 60 having a first portion 60a located in the body 10a of the aneurysm, and a second portion 60b deployed across the neck 12 of the aneurysm and abutting a portion of the inner wall 14b of the artery 14 adjacent the neck. For the purposes of description, the device 60 is described as having wire mesh components although it is understood that other materials having appropriate biocompatibility and structural properties may be utilized. These may also include bio-absorbable components that remain structurally strong for a period of time sufficiently long to enable clot formation in the aneurysm but thereafter may lose that integrity and break down. Different parts of the occlusion device may have different bio-absorbability.

The first portion 60a preferably comprises thin flexible wire filaments that are interwoven into a mesh that is formed into a spherical shape, eg. a wire mesh ball. The diameter and density of the wires, the size and shape of the spaces between the interwoven wires, and the size of the mesh ball are manufactured in accordance with known procedures and that allow conveyance to the aneurysm in a compressed state within a catheter.

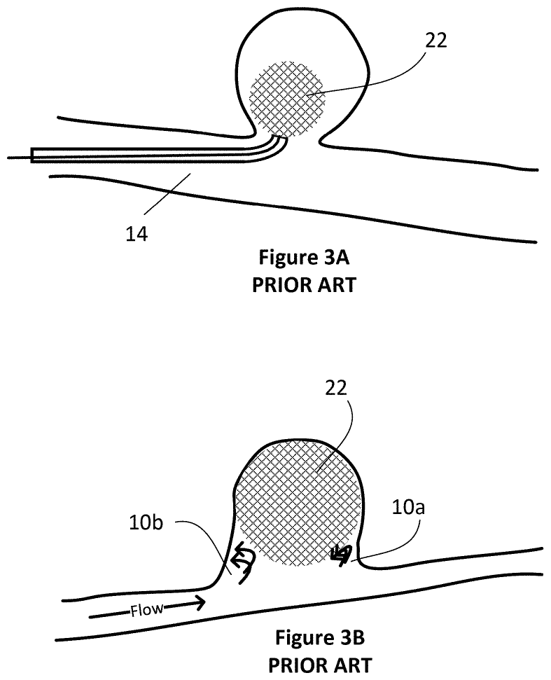

The second portion 60b of the occlusion device 60 is a flexible bridging segment that covers the neck 12 of the aneurysm and is also preferably made of wire mesh, a wire mesh coated with a non-thrombogenic material or a bio-absorbable material. In certain embodiments, the second portion comprises at least one layer of an interwoven mesh of wire filaments, defining a thin disk. The second portion is preferably formed in the shape of a circle or an ellipse, as can be seen in FIGS. 6C (bottom view) and 6D-6M but also being flexible to abut along the inner curved wall 14b of an artery 14 and otherwise create a smooth and flexible surface. FIG. 6C illustrates the second portion as circular (shown in a “wrapped” position within an artery and hence appearing truncated), however the second portion can be of various shapes, such as circular, oval or irregularly shaped and/or include a plurality of individual leaves extending outwardly from a central connection point 60c. The second portion of the occlusion device is preferably attached to the first portion at connection point 60c by weaving or spot welding the portions together, or by using another suitable attachment mechanism. When in position, the occlusion device prevents or slows the flow of blood into the aneurysm, thereby allowing a thrombus to form in the aneurysm. Unlike a wire mesh ball as shown in FIG. 3B, the entire neck of the aneurysm is covered by the second portion thereby preventing areas of turbulence.

Importantly, both the first and second portions are manufactured with shape memory that enhances placement of the device in a variety of anatomical situations. For example, in one embodiment, the first portion is a wire mesh ball that when expanded will assume a generally spherical shape in its relaxed/static position. As such, any inward deformation of the ball will create a force opposing the deformation.

The second portion can be manufactured enabling it to assume different shapes in its relaxed/static position which can be useful in ensuring that the occlusion device remains fixed within the aneurysm. For the purposes of description, the second portion can have both an x and a y axis (FIG. 6C) and will have a generally circular or elliptical shape when viewed from above. In various embodiments, pre-formed curves may be incorporated into the second portion about the x or y axis to enhance positioning and anchoring the device within an aneurysm and to provide effective fitting for particular anatomical configurations. Generally, the pre-formed curves will be biased towards the first position.

In other embodiments, the second portion is a flat circular disk 65 having a plurality of leaves or segment arms 65a surrounding a central core 66. In this embodiment, cuts 67 extend from the perimeter of the circular disk towards the central core. Creases 68, at the perimeter of the central core may be included to act as fold lines allowing each segment arm 65a to flex up or down as shown in FIG. 6F when positioned. As shown in FIG. 6E, spaces 69 may exist between each segment arm to not overlap with each other when bent. Generally, as shown in FIG. 6F, the central core 66 will be sized to completely cover the neck of an aneurysm whereas the segment arms will flex against the interior wall of the artery 14. In this regard, in its relaxed state, the individual segment arms 65a will be biased in an upward direction (i.e. towards the first portion) as denoted by 70 in FIG. 6G. An upward bias will ensure engagement of the segment arms when positioned. In addition, each arm will have appropriate flexibility including torsional flexibility to enable an arm to smoothly fit against an artery wall along different axes and otherwise in all directions.

FIG. 6H shows a schematic three-dimensional view where the individual segments are independently displaceable with respect to one another. Generally, however, it should be noted that while each of the segment arms are shown as planar, due to the relative thinness of each arm, each may flex to conform to the artery curvature and/or other 3D surfaces. In addition, while the crease lines are shown as straight, they may also be curved as depending on the particular flexure properties of the second portion as constructed.

In embodiments shown in FIGS. 6I and 6J, the first portion may also be a partial-sphere or disk having a shape similar to that shown in FIG. 6D or 6E, namely a series of radial segments 67 extending outwardly from the connection point 60c. This design may be advantageous in reducing the overall amount of materials of the occlusion device which may be advantageous for both ease of deployment and retraction as explained in greater detail below. In addition, as radial segments 67 of the first portion primarily serve to hold the second portion in place rather than seal the neck 12, these first portion segments do not need to overlap and/or abut one another as shown schematically in FIG. 6J in top view.

Generally, modest deformation of a lower surface of the first portion will tend to push the first portion into the aneurysm when the deformation is pushing against a lower or side interior surface of the aneurysm. Similarly, modest deformation of the second portion against the curvature of an artery will pull the first portion away from the aneurysm. Thus, these opposing forces will tend to hold the occlusion device within the aneurysm as denoted by the arrows in FIG. 6I.

In further embodiments, as shown in FIGS. 6K and 6L, the segmented portions of the second part may overlap with one another, thus preventing the creation of gaps between individual segments and instead having an overlapped portion 65b. FIG. 6K shows a design with 8 segments 65a and FIG. 6L shows a design with 4 segments 65a. Generally, overlapping segments will range from 4-8. As shown, the segments will create the overlap zone between the central position 60c and the diameter of the neck opening 12 (shown as a round circle in dotted lines). A portion 65c will extend beyond the diameter of the neck opening when deployed. Thus, to the extent that one or more segments flexes to a different extent compared to an adjacent segment, the two segments may slide with respect to one another without creating a gap. Depending on the shape of the aneurysm and particularly for longer elliptical-type aneurysms, after deployment a segment may also be deflected inside the aneurysm if it cannot engage with an edge of the neck.

Moreover, each zone of a segment (i.e. an inner zone 65d and an outer zone 65c) may be provided with different wire mesh opening sizes. For example, as the inner zone is intended to seal, the inner zone may have a tighter mesh compared to the outer zone. The radial segments will generally have a tear-dropped or “petal” shape.

Overall, the occlusion device is anchored in place by the properties of the first and second portions. If the first portion is an outwardly expanding sphere or partial sphere/ellipse and similar in size to the aneurysm, the outward pressure of the first body against the lower inner walls of the aneurysm body helps hold the first body in place in the aneurysm body. Upwardly biased arms of the second portion will ensure contact with the artery walls and hence create a smooth surface for blood flow.

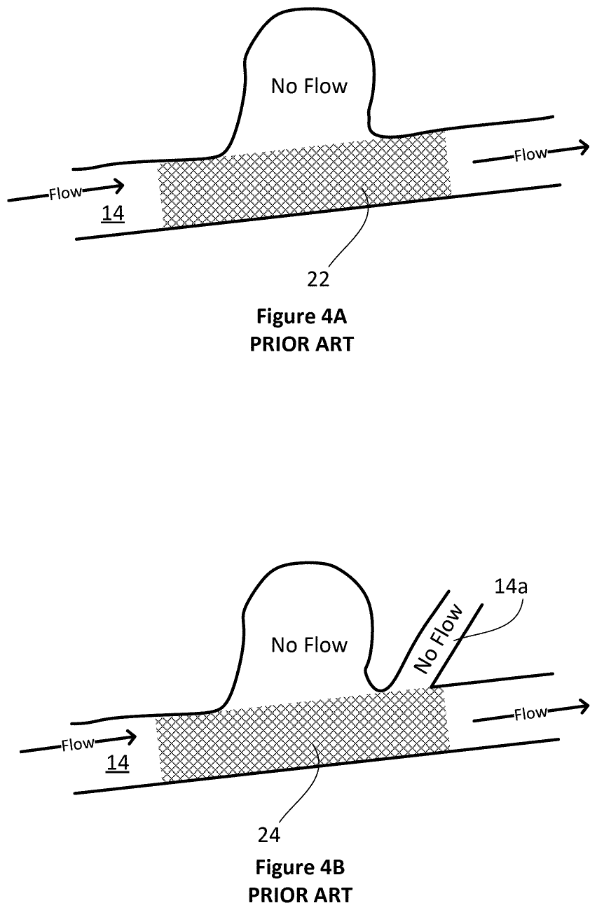

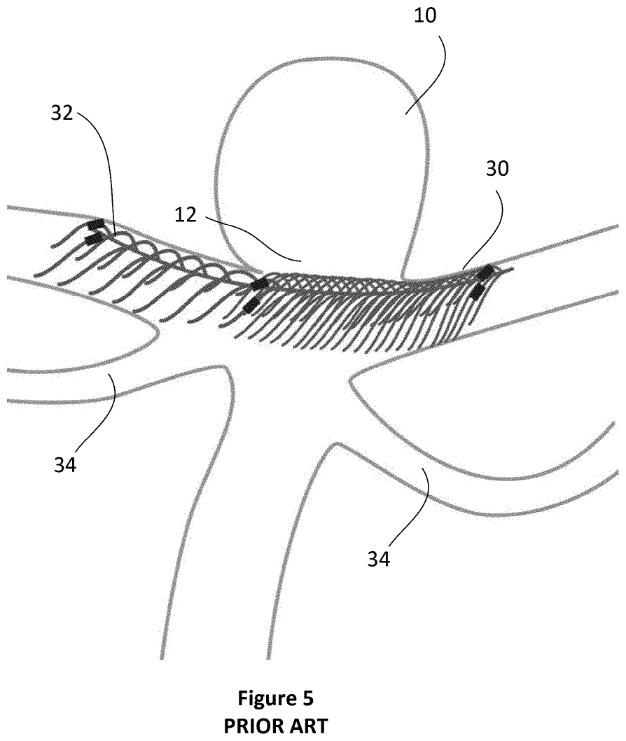

Preferably, the occlusion device would be stable within an aneurysm due to the outward/downward pressure exerted against the inner aneurysm walls. However, in the case of wide necked or highly irregular aneurysms where there is insufficient friction to hold the first part in place (and since the second part is trying to collapse towards the first part and is as a consequence ‘pulling’ the first part out of the aneurysm), in some situations, there may be the need for a tubular stent (similar to stent assisted coiling) to hold the device in place similar to the process as shown in FIG. 4A. In this case, however, a shorter stent 100 (FIG. 6M) may be deployed and may only be required on one side of the aneurysm thus significantly reducing the overall amount of metal in contact with blood. In other words, since the second portion 60b of the occlusion device only covers a portion of the inner wall 14b of the artery and does not cover the entire circumference like a stent does, and is only minimally in the parent vessel, it is likely to be dramatically less thrombogenic and hence may reduce the need for antithrombotic agents. Such stents may also be bio-absorbable in some circumstances.

Further, a stent 100 may be constructed with relatively larger openings, as the stents primary purpose is support as opposed to sealing, and hence utilize less metal.

FIGS. 6N-6Q show embodiments of mechanisms to ensure that the leaves of the second portion 60b are biased upwards after deployment. FIG. 6N shows a mechanism of deployment where the leaves of the second portion are deployed from a microcatheter 30 and where the leaves of the second portion are initially loaded in the microcatheter in a proximally facing orientation (dotted lines). Upon deployment by a microwire/push device 32 (explained in greater detail below) the leaves of the second portion are biased upwardly to a relaxed/static position as shown by the solid lines 60b. FIG. 6O shows an enlarged region of FIG. 6N showing the connection point 60c between the microwire, first portion and second portion in both the collapsed state (dotted line) and deployed state (solid line). The connection point 60c includes a portion 60c′ that remains attached to the microwire/push device 32 after deployment. As shown, the first portion is bonded to the connection point as are the individual leaves of the second portion. The microwire is detachably configured to the connection point at the junction between 60c and 60c′.

In the embodiment shown in FIGS. 6N and 6O, the first portion 60a is bonded to a distal end of the connection point 60c and the second portion (i.e individual leaves 60b) are bonded to an outer surface of the connection point 60c. In order to provide the biasing force to move the leaves to the relaxed/static position (solid lines), an inner portion of each leaf may be provided with a flexure zone 61 having shape memory to bias the collapsed leaves 60b (dotted lines) to the expanded position. That is, the flexure zone 61 will be manufactured to move towards the relaxed position when unconstrained due to internal spring memory. That is, each radial segment will generally want to move to a position upward of a plane tangential to a base of the first portion.

In the embodiment as shown in FIGS. 6P and 6Q, the upward biasing force may be provided the orientation of the attachment of the leaves to the connection point 60c. In this embodiment, the connection point may be a sleeve and where the ends of the first portion and leaves are inserted into the distal end of the sleeve and bonded within the sleeve. In this case, the upward biasing force will be provided the spring forces within the leaves tending to move the leaves in the distal direction.

It is expected that those skilled in the procedure, could place the second part eccentrically over the neck of the aneurysm by manipulating the tip of a microcatheter (if the tip of the microcatheter is not centrally placed in the neck) in which case the second part would be deployed eccentrically. This would be specifically useful in situations where there is a known important vessel just on one side of the aneurysm e.g. anterior choroidal artery. For example, if the aneurysm had a neck diameter of 8 mm and the diameter of the second portion was 14 mm (hence extending 3 mm on both sides of the aneurysm, the physician may place the device such that the second portion overlaps with the artery with 1 mm on one side and 5 mm on the other side. Radio-opaque markers on the first and/or second portions may be effective to guide the physician with this positioning.

Importantly, by having the second portion 60b of the occlusion device cover the neck of the aneurysm, the occlusion device is suitable in aneurysms having wide-necks, and aneurysms having an obtuse neck angle as shown in FIG. 10, since the second portion 60b helps retain the first portion 60a in the aneurysm body.

As noted, various portions of the occlusion device may include one or more radio-opaque portions to assist the surgeon in the deployment, positioning and verification of position during a procedure.

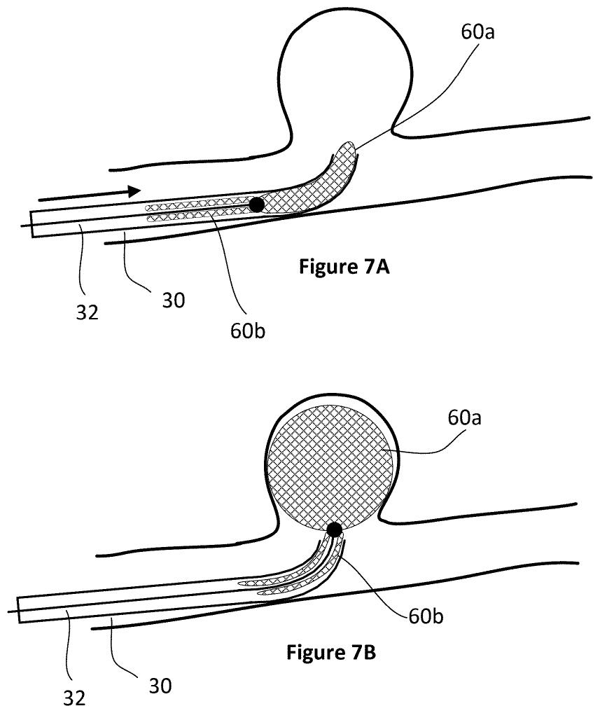

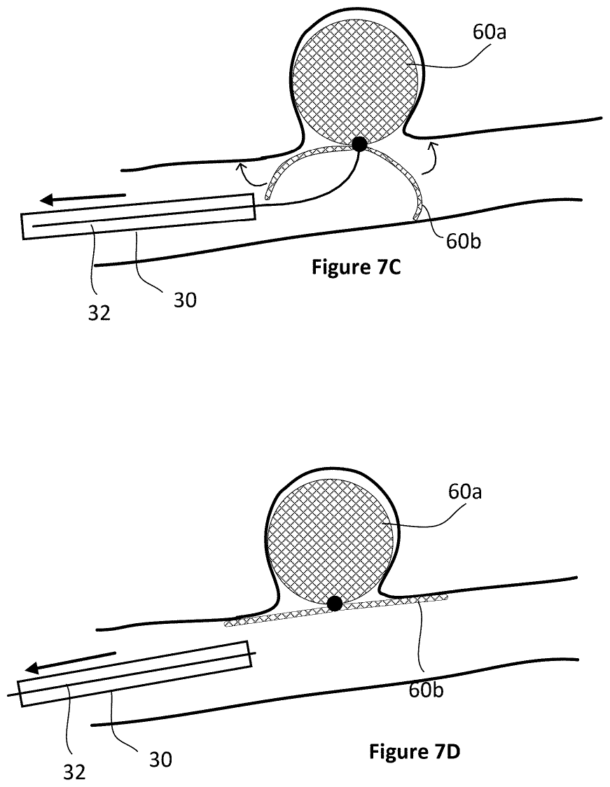

FIGS. 7A to 7D illustrate the deployment of occlusion device 60. A microcatheter 30 is inserted into a patient's arterial system, typically through the groin, and threaded through the vascular system to the site of the brain aneurysm 10, shown in FIG. 7A. Various techniques may be employed to advance the microcatheter to an appropriate location including the use of various combinations of guide catheter, distal access catheters, and diagnostic catheters as known to those skilled in the art. Generally, a physician will choose an occlusion device having an appropriate size and features for the observed size and structure of the aneurysm and nearby anatomical features. As such various combinations of first and second portions may be combined by a manufacturer to provide the physician with a number of different choices for the particular aneurysm. For example, an eccentrically inclined aneurysm may be best fit with an ellipsoid shaped first portion. Accordingly, different combinations of dimensions of devices will ideally be available to the physician including variations in the key parameters of first portion diameter/length/structure and second portion diameter/length/structure. Preferably, each device will be available in a kit form including the attached microwire and encapsulating microcatheter such that the physician can save time after determining which device to use by not having to assemble the system during a procedure.

During the process of deployment, the occlusion device 60, including the first portion 60a and the second portion 60b, is collapsed inside the microcatheter near the distal tip 30a of the microcatheter, and attached to a guide wire 32 that extends all the way to and beyond the proximal tip of the microcatheter at the site of entry into the patient's vascular system. Alternatively, the guide wire and occlusion device can be threaded into the microcatheter from the proximal end to distal tip after the microcatheter is in place in the arterial system.

Once advanced to the site of the aneurysm, the first portion 60a of the occlusion device 60 is pushed out of the distal tip 30a of the microcatheter by pushing the guide wire further into the microcatheter from the proximal end. As the first portion 60a is released into the aneurysm body 10, it expands to its preformed and expanded state, which is typically a sphere, and fills or at least partially fills the body of the aneurysm, as shown in FIG. 7B. At this point, the second portion 60b of the occlusion device is still collapsed in the microcatheter. The position of the first portion 60a of the occlusion device within the aneurysm can be slightly adjusted by moving the microcatheter as needed. Alternatively, if the first portion is not in the correct location, it can be retracted back into the microcatheter by pulling back the guide wire, repositioning the microcatheter and again pushing out the first portion of the occlusion device into the aneurysm body. Or, if it is realized that the first portion of the occlusion device is not the right size and/or shape for the aneurysm, or there are other problems, the first portion can be retracted and the entire occlusion device and possibly the microcatheter can be removed from the artery.

After the first portion 60a of the occlusion device is satisfactorily deployed in the aneurysm body, the second portion 60b of the occlusion device can be deployed by retracting the microcatheter, causing the second portion 60b to exit the distal tip 30a of the guide wire, as shown in FIG. 7C, and expand into its expanded shape, that extends across the aneurysm neck 12 and abuts the inner wall 14 of the artery next to the aneurysm neck. Again, if the positioning of the second segment is not satisfactory, or another problem is encountered, the second portion 60b, with or without the first portion 60a, can be retracted back into the microcatheter using the guide wire and either redeployed or retracted completely out of the body. The use of another catheter such as a distal access catheter may be advanced over the microcatheter in some situations to assist in pushing the second portion into position.

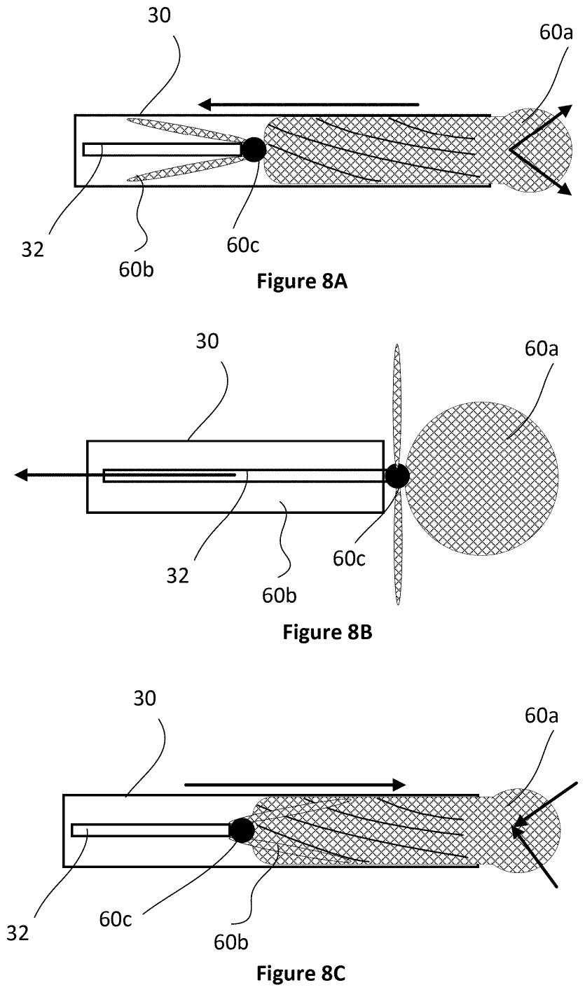

As shown in FIGS. 8A-8C, depending on its design, the second portion may “invert” and return into the microcatheter overlapped with the first portion. FIG. 8A shows schematically how the first and second portions may be held within a microcatheter 30 while connected to a microwire 32. At this stage, the second portion 60b is extending proximally relative to the connection point 60c. If a problem is encountered and the occlusion device needs to be withdrawn (FIG. 8B), the second portion will engage with distal edge of the microcatheter, invert and be withdrawn back into the microcatheter (FIG. 8C). In this case, the microcatheter would likely have to be fully withdrawn and the device “repacked” to the configuration shown in FIG. 8A prior to be re-deployed. An appropriate and separate re-packing device may be required to complete this (not shown).

After deployment of the occlusion device 60, the occlusion device is separated from the guide wire using any suitable means as known to those skilled in the art. For example, a micro-current can be sent through the guide wire to cause the occlusion device to break off the guide wire. The microcatheter can then be removed from the artery.

In one embodiment, the distal edges of the second portion may also be attached to one another (not shown) and/or the microcatheter with a breakable connection which only breaks (passively or actively) as the distal edges are deployed from the microcatheter. This may facilitate proximal movement of the device within the microcatheter during the deployment procedure if necessary.

Although the present invention has been described and illustrated with respect to preferred embodiments and preferred uses thereof, it is not to be so limited since modifications and changes can be made therein which are within the full, intended scope of the invention as understood by those skilled in the art.

1.-24. (canceled)

25. A device for inserting into a cerebral aneurysm to occlude blood flow into the cerebral aneurysm, the cerebral aneurysm having a neck opening and a plurality of walls adjacent the neck opening, comprising:
a first portion for inserting into the cerebral aneurysm to occlude blood flow into the cerebral aneurysm from a cerebral blood vessel, the first portion having an expandable and compressible mesh having dimensions for insertion into and expansion against the cerebral aneurysm walls;
a second disk portion having a flexible, collapsible mesh connected to an outer surface of the first portion at a connection point and having dimensions for covering an outside circumference of the neck opening, where the second disk portion includes a plurality of radial segments that can independently flex relative to each other;
where the combination of the first portion and the second disk portion have a combined resilient flexibility to effectively bias the radial segments of the second disk portion against the neck opening in a substantially flat manner and parallel to the cerebral vessel wall when the first portion is engaged within the cerebral aneurysm.

25. The device as in claim 25 where the second disk portion includes a central core connected to the connection point and the second disk portion radial segments are connected to and surrounding the central core.

26. The device as in claim 26 where the central core has dimensions to substantially cover the neck opening.

27. The device as in claim 25 where the device is reversibly collapsible and expandable into and from a microcatheter.

28. The device as in claim 28 where the device is selectively detachable from a microwire/pusher wire within the microcatheter.

29. The device as in claim 25 where the first portion is a sphere or an ellipsoid.

30. The device in claim 25 where the first portion is a half sphere or half ellipsoid.

31. The device as in claim 25 where the first portion is a wire mesh.

32. The device as in claim 25 where the second disk portion is circular.

33. The device as in claim 25 wherein the second disk portion includes 4-8 radial segments.

34. The device as in claim 34 wherein the radial segments partially overlap with respect to one another.

35. The device as in claim 25 where the second disk portion is a bio-absorbable material.

36. The device as in claim 25 where the second disk portion is collapsible within a microcatheter in an inverted position.

37. The device as in claim 25 where each radial segment has a flexure zone adjacent the connection point having a shape-memory to bias each radial segment into an extended position upward of a plane tangential to a base of the first portion.

38. The device as in claim 38 wherein the shape-memory of the flexure zone enables each radial segment to be loaded into a catheter with the radial segments oriented in a proximal direction and when loaded each radial segment is biased against an inner wall of the catheter and where upon deployment of the device from the catheter, the flexure zone of each radial segment biases the radial segments to the extended position.

39. The device as in claim 25 where the connection point includes a sleeve having a proximal end and distal end and where the first portion and second portion are secured to the connection point through the distal end so as to extend distally from the connection point.

40. The device as in claim 25 where the first portion includes a series of radial segments extending outwardly from the connection point.

41. A kit for conducting a medical procedure to treat an aneurysm comprising an occlusion device as described in claim 25 operatively connected to a microwire and operatively collapsed within a microcatheter.
